(19) United States
(12) Patent Application Publication
(10) Pub. No.: US 2025/0180004 A1
(43) Pub. Date: Jun. 5, 2025

(54) LIQUID DISTRIBUTION SYSTEM

(71) Applicant: ALIVA AS, Kristiansand S (NO)

(72) Inventors: Preben Falkum, Kristiansand (NO); Torgeir Bråtane, Kristiansand (NO); Sindre Sørhus, Kristiansand (NO)

(21) Appl. No.: 18/842,258

(22) PCT Filed: Feb. 27, 2023
(86) PCT No.: PCT/EP2023/054879

§ 371 Date: Aug. 28, 2024

(30) Foreign Application Priority Data
Mar. 1, 2022 (GB) 2202833.6

(51) Int. Cl. F04B 43/12; A61M 5/142

(52) U.S. Cl. CPC F04B 43/1284; A61M 5/14232; F04B 43/1276

(57) ABSTRACT

A tube bracket for operatively engaging a tube with a peristaltic pump, the tube bracket comprising: a housing defining: a pump cavity for receiving a roller assembly of the peristaltic pump; and a tube path for receiving the tube, the tube path extending at least partially circumferentially around the pump cavity; and a part configured to receive a force such that, in use, a portion of the tube received in the tube path is biased against the roller assembly of the peristaltic pump in the pump cavity. A replaceable tube assembly comprising the tube bracket and a liquid distribution system comprising the tube assembly is also provided.

### CROSS-REFERENCE TO RELATED APPLICATIONS

This application is a 35 U.S.C. 371 national phase filing of International Application No. PCT/EP2023/054897 filed on Feb. 27, 2023, which claims priority to United Kingdom Patent Application No. 2202833.6 filed on Mar. 1, 2022, wherein the entire contents of the foregoing applications are hereby incorporated by reference herein.

### TECHNICAL FIELD

The technology described herein relates to liquid distribution. In particular, a tube bracket for operatively engaging a tube with a peristaltic pump is disclosed. Furthermore, systems and methods for distributing liquid are disclosed, using a peristaltic pump and a tube bracket for operatively engaging a tube with the peristaltic pump.

### BACKGROUND

Xerostomia is a dry mouth symptom associated with insufficient production of saliva, which is commonly seen in elderly people as a possible side effect of many types of medication. As saliva has a plurality of functions in the mouth, xerostomia may lead to many connected symptoms, for example dental caries, acid erosion of the teeth, difficulties with swallowing food, etc.

Xerostomia can have different causes, and treatment of the underlying cause is often difficult. Thus, treatment is mostly concerned with alleviating the symptom rather than curing the cause. A known treatment for xerostomia is to deliver fluids orally in a continuous manner, or when desired by the patient, with the help of a device placed in the user's mouth through which fluid from an external source is pumped. Such devices have the advantage of mimicking the production of saliva for the salivary glands.

Peristaltic pumps are advantageously able to pump clean/sterile fluids along tubing without exposing those fluids to contamination from exposed pump components, which makes them suitable for a wide range of medical applications. Peristaltic pumps comprise a roller assembly having rollers or shoes that compress the tube as they rotate, which creates a vacuum and draws fluid through the tube.

However, tubing that is being pumped by a peristaltic pump tends to degrade over time as a result of the pumping action and needs to be periodically replaced. Moreover, tubing and associated components may need to be regularly replaced for hygiene reasons. Accordingly, they are not ideal for use in home medical devices where the user/patient is required to perform this tubing replacement themselves, which can be an awkward procedure. For example, it is difficult to ensure that the tube is positioned correctly relative to and suitably compressed against the roller assembly of a typical peristaltic pump, particularly for frail and/or elderly users. Moreover, it can be difficult to maintain hygienic conditions while using them.

Thus, it would be advantageous to provide a peristaltic pump and tubing assembly which simplifies the process of administering liquid to a user.

### SUMMARY

A first embodiment comprises a tube bracket for operatively engaging a tube with a peristaltic pump, the tube bracket comprising: a housing defining: a pump cavity for receiving a roller assembly of the peristaltic pump; and a tube path for receiving the tube, the tube path extending at least partially circumferentially around the pump cavity; and a part configured to receive a force such that, in use, a portion of the tube received in the tube path is biased against the roller assembly of the peristaltic pump in the pump cavity.

The tube bracket as described allows a user to easily engage a tube with a peristaltic pump in an effective manner. In other words, with the use of the tube bracket a user can insert a tube into a tube path and secure the tube in a first operation, before fitting the tube bracket onto a roller assembly of a peristaltic pump in a second operation, and then applying a force to the tube bracket in a third operation such that a portion of the tube is correctly biased against the roller assembly for effective operation. Thus, the tube bracket breaks the process of replacing a tube in a liquid distribution system (such as a medical liquid distribution system) comprising a peristaltic pump into simple, feasible steps, even for those users with reduced fine motor skills (e.g. the elderly). In fact, the first operation of fitting the tube into the tube path may be carried out in advance, for example this may be done by a manufacturer. The user will then only need to carry out the second and third operations. This is discussed further below.

A tube as described herein may also be termed a hose.

It will be appreciated that a tube being biased against the roller assembly means that it is pushed or pressed against the roller assembly.

Advantageously, the part of the tube bracket is particularly configured to receive a force, such that, in use, a portion of the tube received in the tube path is biased against the roller assembly of the peristaltic pump in the pump cavity. In other words, when the peristaltic pump is operating to effect a flow of liquid through the tube, the tube bracket engages the tube with the roller assembly of the peristaltic pump by means of the received force. The portion of the tube received in the tube path may therefore be biased by that received force. For example, a force received by the part may move the tube bracket towards the roller assembly of the pump thereby biasing, for example pressing, a portion of the tube received in the tube bracket against the roller assembly. Therefore a force received by the part may cause the bracket to move into an operational position such that a portion of a tube received in the tube path is pressed against a roller assembly.

A force application mechanism may therefore apply a force to the bracket during use of the pump. The received force may move the bracket into an operational position in which the bracket biases the tube against the roller assembly. The bracket may be held in the operational position by the received force. In that operational position a force may therefore continue to be received by the part.

Alternatively, once the received force has moved the bracket into an operational position the bracket may be held in the operational position by a mechanism, for example by engagement with a notch in a housing of the pump or by a latching mechanism, such that a force no longer needs to be received. However the tube will continue to be biased against the roller assembly by virtue of the bracket being in the operational position. It will be appreciated that in all these scenarios, the biasing of the tube against the roller assembly is a result, whether directly or indirectly, of a force being received by the part.

The force which the part is configured to receive may be an external force, which may be applied by a user or may be a force applied by an external mechanism, i.e. from a mechanism not part of the tube bracket, which has been operated by the user. Omitting any complicated mechanisms from the tube bracket enables the tube bracket to be a single-use, i.e. disposable, item that can be easily replaced, and avoids the tube bracket being difficult for those with reduced fine motor skills (e.g. the elderly) to use.

In other words, the tube bracket may be a monolithic structure, in that it is a simple, unitary article. The tube bracket may be formed from a single material. The tube bracket (that is, the housing and the part configured to receive a force) may be integrally formed, for example as one piece, or the part configured to receive a force may be permanently attached to the housing. The part configured to receive a force may be fixed relative to the housing such that it cannot move relative thereto.

The tube bracket may comprise or be made from a plastic material. It may be manufactured by injection moulding, i.e. it may be injection moulded. In one example, the tube bracket may comprise or be made from polypropylene copolymer (PP-C). This provides good mechanical properties. The tube bracket may alternatively comprise or be made from any one or combination of the following: polyamide (PA), polystyrene (PS), polycarbonate (PCT), Acrylonitrile butadiene styrene (ABS). Glass may also be included to improve form stability and reduce cost. Recycled plastic may also be utilised.

Preferably the tube bracket is substantially rigid such that no significant bending takes place when a force is applied in use.

The part of the tube bracket configured to receive a force may also be termed a portion of the tube bracket. Therefore it may be considered as a force receiving portion. The part may be an element, member, or feature of the tube bracket.

The part of the tube bracket configured to receive the force may be a brace and/or comprise a reinforced structure. For example, the reinforced structure may be a thickened and/or more rigid portion of a wall of the housing.

In an embodiment, the part may be a protrusion which extends outwardly from the housing.

Thus, the protrusion may be relatively large compared to the housing of the tube bracket, which makes it easy to apply a (external) force thereto. Optionally, the protrusion comprises a flange extending outwardly from the housing. The flange may comprise a crest shape.

At least part of the protrusion may be configured to be easily held by a user when the tube bracket is fitted onto the roller assembly of the peristaltic pump, in other words the protrusion may also be a convenient handle by which the user can grip the tube bracket. Alternatively, the housing may comprise a handle separate to the protrusion.

The tube which the tube bracket is for operatively engaging may typically be a flexible tube. However, it is also envisaged that a tube may have a rigid portion pre-formed with a complementary shape to the tube path into which it is to be received.

Optionally, the tube path comprises a shoulder portion configured to, in use, position the portion of the tube to be biased closer to the roller assembly than the rest of the tube.

The tube path may be defined by an external wall. The external wall may be thicker over a certain length to define a shoulder portion. Therefore, the portion of the tube which sits adjacent to the shoulder portion may be positioned radially closer to the centre of the pump cavity of the tube bracket than portions of the tube in the rest of the tube path.

Optionally, the position of the shoulder portion of the tube path is proximate to the part of the tube bracket where the tube bracket is configured to receive the applied force.

In use, the force may be applied to the tube bracket in a radial direction towards the roller assembly of the peristaltic pump (in order to bias a portion of the tube against the roller assembly). By positioning a shoulder portion proximate to where the tube bracket is configured to receive the application of force (for example, circumferentially aligning, with respect to the roller assembly, the shoulder portion and the protrusion), the portion of the tube to be held against the roller assembly may be even more effectively compressed.

Optionally, the tube bracket comprises a bracket tab extending outwardly from the tube bracket in a radial direction of the pump cavity.

The bracket tab may be configured to fit into a corresponding slot of a housing of the peristaltic pump when the tube bracket is fitted onto the roller assembly of the peristaltic pump.

Thus, in use, the bracket tab is inserted into the slot when the tube bracket is fitted onto the roller assembly of the peristaltic pump, and thus helps to prevent the tube bracket from rotating when the roller assembly is moving.

The part configured to receive a force may comprise the bracket tab. For example if the part configured to receive a force is a protrusion, the protrusion may comprise the bracket tab. In that case the tab may be termed a protrusion tab.

Optionally, the tube bracket comprises a housing tab extending outwardly from the tube bracket housing in a radial direction of the pump cavity.

The housing tab may be configured to fit into a corresponding slot of a housing of the peristaltic pump when the tube bracket is fitted onto the roller assembly of the peristaltic pump. The housing tab may extend in a radial direction opposite to the bracket tab.

Thus, in use, the housing tab is inserted into the slot when the tube bracket is fitted onto the roller assembly of the peristaltic pump, and thus helps to prevent the tube bracket from rotating when the roller assembly is moving. Optionally, the tube bracket comprises one or more clips configured to secure the tube in the tube path. There may be a bend at the end of each clip to provide guidance for a tube received therein where it leaves the bracket.

The housing of the tube bracket may comprise a pair of arms, such as straight arms, extending away from the pump cavity. The tube path may extend along these arms. The clips may be located on these arms. The tube path of one of the arms may be configured to guide the tube towards the pump cavity from a container and the tube path of one of the other arms may be configured to guide the tube away from the pump cavity.

It will be appreciated that all of the features described above in relation to the tube bracket and its use are equally applicable to the various systems, assemblies and methods described below. In particular, features described in relation to the tube bracket in use with a peristaltic pump are particularly relevant to the liquid distribution systems and methods described below and thus will not be repeated again there.

A second embodiment comprises a tube assembly for a liquid distribution system, the tube assembly comprising: a tube for liquid communication; and a tube bracket according to the first embodiment, optionally comprising any of the optional features described above. The tube assembly may be a replaceable tube assembly.

Advantageously, the tube assembly may be a single-use, i.e. disposable, item, that can be easily replaced in a liquid distribution system. Typically, the tube assembly may be suitable for use for up to 24 hours. However, it may be suitable for a longer time period (for example 36 hours, 48 hours or longer). At the end of that time, it would generally be disposed of and replaced with a new tube assembly. This provides the benefit that it is not necessary to clean the tube assembly to keep good hygiene. The simple design of the tube assembly makes it cost-effective to manufacture, whereby single use is feasible for the user and/or manufacturer.

Optionally, the tube is preassembled in the tube bracket. In other words, in the replaceable tube assembly the tube has already been fitted/secured into the tube bracket. Advantageously, this means that a user will not have to fit the tube into the tube path of the tube bracket themselves, which might be difficult and/or awkward to perform, particularly for older users.

Optionally, the tube assembly comprises a medical applicator device, wherein the medical applicator device is configured to receive liquid from the tube and administer it to a user.

In an embodiment, the medical applicator device is a mouthpiece for the user's mouth. The mouthpiece is configured to deliver fluid orally to the user, particularly for the treatment of xerostomia. Alternatively, the medical applicator device may be a catheter configured to deliver fluid to the body of the user. The liquid/fluid may comprise vitamins, medication, nutrients or other chemicals.

The tube may be fluidly coupled with the medical applicator device. For example, it may be connected to or integrally formed with the medical applicator device. For example, in the case of a mouthpiece, the mouthpiece end of the tube may be reversibly or irreversibly connected to the inlet of the mouthpiece. However, preferably, the tube is irreversibly connected, i.e. permanently fixed, to the inlet of the mouthpiece. The tube and mouthpiece may be integrally formed. This reduces the likelihood of a user accidentally removing the tube from the inlet of the mouthpiece.

Optionally, the tube assembly comprises a container for containing the liquid to be distributed. The tube may be fluidly connected to the container.

The container end of the tube (i.e. the end opposite to the medical applicator device) may be configured to be easily attachable to and detachable from the container. For example, the container end may be secured in a seal in a screw top for the container.

The tube may comprise a first tube section connected to the medical applicator device and a second tube section connected to the container.

The tube may comprise a check valve joining the first tube section to the second tube section. A check valve may inhibit liquid from the medical applicator device flowing back into the second tube section and/or the container. Thus, the hygiene of the assembly is improved.

Alternatively, the tube assembly may not include a check valve and the tube may comprise a simple connector joining the first tube section to the second tube section. Thus, the tube assembly is sufficiently simple and cost-effective to manufacture.

The second tube section, which may be the tube section received in the tube bracket, may comprise thicker tubing than the first tube section. Advantageously, this reduces the risk of tubing rupture from the action of the peristaltic pump, thus improving the reliability of the tube assembly.

For example, the first tube section may be from 1 m to 3 m long, for example 2m long. Alternatively, the first tube section may be between about 10 cm and 70 cm, between about 30 cm and 50 cm, and may be about 40 cm. The first tube section may have an outer diameter of between 1.0 mm and 3.0 mm, and may have an outer diameter of about 2.0 mm.

The second tube section may have an outer diameter of between 1.0 mm and 5.0 mm, and may have an outer diameter of about 4.0 mm. The second tube section may be any suitable length. For example, it may be between about 10 cm and 100 cm, between about 40 cm and 70 cm, and may be about 60 cm. The second tube section may be shorter than the first tube section.

The tube may comprise an elastomeric material, such as silicon.

The container may be configured for containing a liquid comprising dissolved carbon dioxide, so that the carbon dioxide stays dissolved while the liquid is inside the container. The container should therefore be sufficiently strong to not expand, and sufficiently air tight to not allow carbon dioxide gas to escape. Carbon dioxide may for example be introduced to the liquid under high pressure. Carbon dioxide in the liquid will cause bubbles to form when the pressure is decreased, for example when the liquid is led from the container to a mouthpiece. The bubbles may function to keep the internal channel and/or the outlets of the mouthpiece clean and may cause the liquid to resemble saliva more, since saliva typically contains bubbles. Carbon dioxide additionally has the benefit that it may make the liquid slightly acidic, which may trigger the production of saliva from the parotic glands in the mouth. The container may therefore comprise a liquid comprising dissolved carbon dioxide.

A third embodiment comprises a tube assembly for a liquid distribution system comprising a tube for liquid communication and a tube bracket for operatively engaging the tube with a peristaltic pump, the tube bracket comprising: a housing defining: a pump cavity for receiving a roller assembly of the peristaltic pump; and a tube path extending at least partially circumferentially around the pump cavity in which the tube is received; and a part configured to receive a force such that, in use, a portion of the tube received in the tube path is biased against the roller assembly of the peristaltic pump in the pump cavity.

The tube bracket of the third embodiment may comprise any or all of the optional features of the tube bracket of the first embodiment.

A fourth embodiment comprises a liquid distribution system comprising: a tube for liquid communication; a peristaltic pump for effecting a flow of liquid through the tube; and a tube bracket configured to operatively engage the tube with the peristaltic pump, the tube bracket comprising: a housing defining: a pump cavity in which a roller assembly of the peristaltic pump is received; and a tube path extending at least partially circumferentially around the pump cavity in which the tube is received; and a part configured to receive a force such that, in use, a portion of the tube received in the tube path is biased against the roller assembly of the peristaltic pump in the pump cavity.

A fifth embodiment comprises a liquid distribution system comprising: a medical applicator device for administering liquid to a user; a container for containing the liquid to be distributed to the medical applicator device; a tube assembly according to the second embodiment (with or without any of the optional features thereof), wherein the tube is fluidly connected to the container and the medical applicator device; and a peristaltic pump assembly for effecting a flow of the liquid from the container to the medical applicator device; wherein the tube bracket is fitted onto a roller assembly of the peristaltic pump assembly.

It will be appreciated that a peristaltic pump assembly comprises a peristaltic pump. Therefore, features of a peristaltic pump are also features of a peristaltic pump assembly. A peristaltic pump assembly may optionally comprise other components related thereto. The peristaltic pump assembly may comprise a housing.

Optionally, the peristaltic pump assembly comprises a mechanism for applying a force to the part of the tube bracket configured to receive a force to bias a portion of the tube against the roller assembly of the peristaltic pump. The mechanism (e.g. a force application mechanism) may be configured such that application of a force to the part moves the tube bracket towards the roller assembly of the pump thereby biasing, for example pressing, a portion of the tube received in the tube bracket against the roller assembly. This may be termed an operational position of the bracket, as discussed above. The operational position of the bracket may be maintained by continued application of a force to the part configured to receive a force. Alternatively the pump assembly may comprise a mechanism configured to maintain the bracket in the operational position (such as a latching mechanism) without the continued application of force as discussed previously above.

The force application mechanism may comprise a member configured to engage the part configured to receive a force. The member may be configured to apply a force to the part. The member may be configured to move, for example push, the tube bracket into the operational position. The member may be operated directly or indirectly by a user.

The mechanism may comprise a handle attached to the member. The handle may be configured to be operated by a user. For example, the handle may be configured to receive a force (including a torque) from a user. The handle may for example be a knob that can be turned. The handle may comprise a grip to be held by the user. The force (including a torque) received from the user may cause the member to apply a force to the part. Accordingly, the handle may be operated to adjust the force applied to the part.

The member may comprise a flange. The member may comprise a disc. Optionally, the member comprises an arcuate portion. This arcuate portion may be configured to adjust the force applied to the protrusion upon rotation of the handle. The member may be substantially D-shaped. The member may comprise a D-shaped disc.

The member may be rotationally mounted to the peristaltic pump assembly. For example, the member may be rotationally mounted to the housing of the pump assembly, optionally by means of a fastener e.g. a screw.

The member may be eccentrically rotationally mounted to the peristaltic pump assembly. The member may be mounted proximate the part configured to receive a force such that rotation of the member causes the member to abut the part. The mechanism may be configured such that further rotation of the member applies a force to the part so as to bias the portion of the tube received in the tube path against the roller assembly. In a non-operational position the member may be disengaged from the part.

The member may be configured to be locked in a position of engagement, e.g. an operational position, with the part of the tube bracket configured to receive a force so as to maintain a force applied thereto. It will be appreciated that by “locked” it is meant that the member may be held or maintained in the position of engagement, for example until a user applies a force to overcome the locking. Therefore the force application mechanism may comprise a means, such as a maintenance mechanism, configured to maintain the member in the operational position such that a force applied to the bracket is maintained and the bracket is maintained in the operational position. Such possible means are discussed further below.

As discussed above, the force application mechanism may comprise a handle, such as a knob, for example a circular or substantially circular knob, coupled to the member. The member may be configured such that rotation of the handle causes rotation of the member. The handle may be rotatable between a non-operational position of the force application mechanism in which no force is applied to the part and an operational position in which a force is applied to the part. It will be appreciated that an operational position of the force application mechanism may correspond to the operational position of the tube bracket, and a non-operational position of the force application mechanism may correspond to a non-operational position of the tube bracket.

As mentioned above, the member may be rotatably mounted by means of a fastener. The handle may be rotatably mounted by means of the fastener. A spring washer may be arranged around the fastener and configured to press against the member on an opposite side to the handle. The member may press against the housing of the pump assembly and hold the handle in place. The fastener may be configured to allow some movement of the handle along the axis of rotation. A plain washer may be arranged around the fastener proximate the spring washer such that if the handle is moved away from the housing along the axis of rotation (e.g. if a user applies a force to the handle) the plain washer will abut the spring washer and transmit the force from the spring washer to the fastener and the knob handle, thus acting to move the handle back along the rotational axis to be seated against the housing.

The mechanism may be configured such that in use, once a user has applied a force (including a torque) to the handle, for example once the user has rotated the handle to the desired position to apply the desired force to the part configured to receive a force, the user can release the handle and the application of the desired force to the part is maintained. For example, the mechanism may comprise a means, such as a maintenance mechanism, configured to maintain the application of the desired force to the part configured to receive a force and/or to maintain the bracket in an operational position

In one example, the maintenance mechanism may be a biasing means which may comprise a spring or a ratchet device configured to maintain the application of the desired force to the part configured to receive a force. In another example, the member may comprise a feature which provides a stable configuration of the member and the part configured to receive a force when the member is applying the desired force to the part. The outline of the member may comprise an indentation, and wherein the part configured to receive a force and the member are in the stable configuration when the part is in the indentation.

The force application mechanism may be configured to maintain the member in the non-operational position until a force is applied by a user to move it to an operational position. The force application mechanism may be configured to maintain the member in the operational position until a force is applied by a user to move it to a non-operational position.

The member may be rotationally mounted and comprise an edge configured to engage and apply a force to the part configured to receive a force, wherein that edge comprises an arcuate portion adjacent a substantially flat portion. For example, the member may be substantially D-shaped. The mechanism may be configured such that rotation of the member engages the arcuate portion with the part configured to receive a force so as to apply a force to the part, and wherein further rotation of the member engages the flat portion with the part. Once the flat portion is engaged a stable configuration is reached such that the flat portion remains engaged with the part configured to receive a force even after the user has released the handle. Consequently, the application of force to the part is maintained.

The force application mechanism may comprise a means (such as a maintenance mechanism) to hold the member in the non-operational position but which may be overcome by a force applied by the user to move it to an operational position. The force application mechanism may comprise a means (such as a maintenance mechanism) to hold the member in the operational position but which may be overcome by a force applied by a user to move it to a non-operational position.

The handle may comprise a protruding portion configured to be seated in a pocket of a housing of the pump assembly when the member is in a position of engagement (e.g. an operational position) so as to lock the member in that position. To explain further, the handle, e.g. a knob, may comprise a protruding portion such as a bump or bulb-shaped member protruding from a surface of the handle (e.g. a surface of the handle adjacent the member). The housing of the pump assembly may comprise one or more, for example two, pockets therein complementary in shape to the protruding portion such that the protruding portion can be seated therein. The protruding portion and a first pocket may be relatively positioned such that in a non-operational position of the force application mechanism the protruding portion aligns with the first pocket and can be seated therein. A second pocket may be positioned such that in an operational position of the force application mechanism the protruding portion aligns with the second pocket and can be seated therein.

The handle may be configured to operate between, such as rotated between, two end positions, for example one end position corresponding to a non-operational position and a second end position corresponding to an operational position. The first pocket may be aligned with the first end position. The second pocked may be aligned with the second end position.

When the protruding portion is seated in a pocket the handle may be maintained in that position, in other words it cannot freely rotate. Thus, a member coupled to the handle may be maintained in that position.

The handle may be fastened with a spring washer that pushes it against the housing of the pump assembly.

The protruding portion may be arranged towards the edge of the handle. It may be located such that when the handle is in a non-operational position, the protruding portion is located laterally horizontally to the left of a rotational axis of the handle. It may be located such that when the handle is in an operational position the protruding portion is located vertically above the rotational axis.

The force application mechanism may be configured such that when a user applies a force to turn the handle, the force applied by the user forces the protruding portion to disengage from one pocket and thus allows the handle to turn. It may further be configured such that when the protruding portion aligns with a pocket, the protruding portion engages that pocket. This engagement maintains the handle in this position. The mechanism may be configured such that the user can feel the protruding portion disengaging/engaging with the pockets (with a “snap”) and thus can feel when the handle is in a non-operational and/or an operational position. Optionally, the tube bracket comprises a tab (a bracket tab) extending outwardly in a radial direction of the pump cavity. The housing of the peristaltic pump assembly may comprise a slot (a bracket tab slot) and the bracket tab may be fitted into the slot. As such, when the member is engaged with the part, the member restricts movement of the bracket tab in the slot. The member may restrict the movement of the bracket tab in a direction of insertion of the bracket tab into the bracket tab slot, e.g. along an axis of the slot. The part configured to receive the force may comprise the bracket tab.

When the bracket tab is fitted into the slot, it helps to prevent the tube bracket from rotating when the roller assembly is moving. When the member engages the part (e.g. the member is in contact with the part), the member lies on top of the tab and helps to prevent the tube bracket from being pulled out of the pump assembly.

Optionally, the tube bracket housing comprises a tab (housing tab) extending outwardly in a radial direction of the pump cavity. The housing of the peristaltic pump assembly may comprise a slot (a housing slot) and the housing tab may be fitted into the slot. The slot may comprise a peripheral recess. The system may thus be configured such that, as the force applied to the protrusion is increased, the tab moves into the recess. The recess may be formed in a side wall of the of the housing slot. The recess may have an axis (e.g. a direction of insertion of the housing tab into the recess) aligned with a direction that force can be applied to the part configured to receive said force.

When the housing tab is fitted into the slot, it helps to prevent the tube bracket from rotating when the roller assembly is moving. When the member engages the part configured to receive a force (e.g. the member is in contact with the part), and the tube bracket is biased in a direction toward the roller assembly, the housing tab is inserted into the recess and helps to prevent the tube bracket from being pulled out of the pump assembly.

The peristaltic pump may be configured to continuously provide fluid in order to simulate a healthy flow of saliva. For example, the pump may be configured to provide fluid flow at a constant rate between 0.0-3.0 m/min, or 1.0-2.0 ml/min. Alternatively, the pump may be precisely controlled by the user to provide fluid as desired. The pump may be configured to reassume a previous setting. Therefore, in the event that a power supply to the pump is terminated, inadvertently or otherwise, upon being reconnected to power the pump can resume its previous operation. The pump may be configured to continuously deliver liquid (e.g. at a constant rate) for more than 2, 4, 6, 8 or 10 hours.

The liquid distribution system may be configured to identify when the tube bracket and/or the container needs to be replaced. The tube bracket and/or the container may include a RFID tag for identification of the tube bracket and/or the container. The pump may comprise a RFID reader positioned/configured to read the RFID tag of a tube bracket and/or a RFID reader positioned/configured to read the RFID tag of a container. The RFID reader(s) may be configured to receive data from the RFID tag relating to the tube bracket and/or container, such as: a type of tube bracket, a type of medical applicator device being utilised with the tube bracket, the period of time that the tube bracket should be used with the container (for example due to hygiene reasons), the internal diameter of the tube held by the tube bracket, or the initial volume of liquid within the container. The period of time may for example be 24 hours, but could be longer depending on the particular use scenario. For example, it could be 36 hours, 48 hours or longer. The liquid distribution system may be configured to stop operation of the pump after the period of time has expired in order to prevent a user from using the liquid distribution system, e.g. to prevent the system being used when it is no longer hygienic.

The liquid distribution system may be configured to perform these actions by means of a control unit. Therefore, the pump assembly may comprise a control unit configured to control the pump, for example it may be configured to perform any of the actions discussed above such as identifying when the tube bracket and/or the container should be replaced. The control unit may be configured to receive input(s) from the RFID reader(s) and determine using these inputs when the tube bracket and/or container should be replaced. The control unit may be provided with information regarding the period of time for which a particular type of tube bracket or particular type of medical applicator device utilised with the tube bracket may be used. The control unit may then for example stop operation of the pump or issue an alert once an allowed time period has expired. The control unit may comprise a processor and a computer readable medium comprising software for carrying out such functions. A time parameter may therefore be set in the software for a particular use scenario.

The pump assembly may be configured (for example by means of a control unit as discussed above) to determine the volume of liquid that has been delivered by the pump to the medical applicator device. To that effect, the peristaltic pump may comprise a step motor and a Hall-effect sensor. Accordingly, the pump assembly may be configured to determine that the volume of liquid remaining in the container is low.

The liquid distribution system may comprise a means (e.g. an alarm or LED) for alerting a user that the tube bracket and/or the container should be replaced (e.g. in response to determining that the volume of water is low, or that the tube assembly has been used for a predetermined period of time).

The liquid distribution system may comprise a control panel configured to enable a user to control various aspects of the system such as a speed of the pump. The control panel may be part of the control unit discussed above or may be a separate control system.

A sixth embodiment comprises a liquid distribution system comprising: a medical applicator device for administering liquid to a user; a container for containing the liquid to be distributed to the medical applicator device; a tube for liquid communication between the container and the medical applicator device; a peristaltic pump for effecting a flow of liquid through the tube; and a tube bracket configured to operatively engage the tube with the peristaltic pump, the tube bracket comprising: a housing defining: a pump cavity in which a roller assembly of the peristaltic pump is received; and a tube path extending at least partially circumferentially around the pump cavity in which the tube is received; and a part configured to receive a force such that, in use, a portion of the tube received in the tube path is biased against the roller assembly of the peristaltic pump in the pump cavity.

The system of the sixth embodiment may comprise any or all of the optional features of the system of the fifth embodiment.

A seventh embodiment comprises a method for distributing liquid to a user, the method comprising: providing a medical applicator device for administering liquid to a user, a container for containing the liquid to be distributed to the medical applicator device, and a tube assembly according to the second or third embodiment (with or without any of the optional features thereof), wherein the tube is fluidly connected to the container and the medical applicator device; providing a peristaltic pump assembly for effecting a flow of the liquid from the container to the applicator device; fitting the tube bracket of the tube assembly onto a roller assembly of the peristaltic pump assembly; applying a force to the part of the tube bracket configured to receive a force to bias a portion of the tube of the tube assembly against the roller assembly; and running the peristaltic pump to effect the flow of liquid from the container to the medical applicator device.

Optionally, applying a force to the part to bias a portion of the tube against the roller assembly comprises operating a mechanism of the peristaltic pump assembly to urge the part toward the roller assembly. Urging the part towards the roller assembly biases the portion of the tube against the roller assembly.

Optionally, the mechanism comprises a handle coupled (e.g. attached) to a member configured to engage the part configured to receive a force and to apply a force thereto. Accordingly, operating the mechanism may comprise manipulating the handle to adjust the force applied to the part (and thus adjust the urging of the tube assembly) toward the roller assembly by means of the member.

Optionally, the member comprises an arcuate portion configured to, in response to rotation of the handle, engage the part and adjust the force applied to the part configured to receive a force. The member therefore urges the tube bracket toward the roller assembly of the peristaltic pump.

Optionally, after the handle has been operated (manipulated) by a user to urge the part toward the roller assembly, the method may include the user releasing the handle whilst the member continues to urge the part toward the roller assembly. In other words, in the method, the mechanism continues to apply a force to the part after the user has released the handle such that the portion of the tube remains biased against the roller assembly. Operating the mechanism may comprise rotating the handle thereby rotating the member to engage the part. For example, it may comprise rotating the handle from a non-operational position in which the member is disengaged from the part to an operational position in which it is engaged with the part and applies a force thereto.

Operating the mechanism may comprise operating a biasing means to maintain the application of the desired force to the part. In one example, the biasing means may comprise a spring or a ratchet device configured to maintain the application of the desired force to the part configured to receive a force, and operating the biasing means may comprise engaging the biasing means with the member. In another example, the member may comprise a feature which provides a stable configuration of the member and the part when the member is applying the desired force to the part. The outline of the member may comprise an indentation, and wherein the part and the member are in the stable configuration when the part is in the indentation. In this example, operating the biasing means may comprise manipulating the handle to move (e.g. rotate) the member such that the part is positioned in the indentation.

The handle may comprise a protruding portion such as a bump or bulb-shaped member protruding from a surface of the handle such that when the handle has been rotated to an engaged position the protruding portion becomes seated in a pocket in a housing of the pump assembly. Accordingly, the handle, and thereby the member, is maintained in the engaged, e.g. operational, position.

Optionally, the handle comprises a flange, for example having a crest shape. This may form a grip for the user to hold. The part configured to receive a force may comprise a flange, having for example a crest shape. The crest shape of the handle may be configured to be aligned with the crest shape of the part when the member is in a maximal position, e.g. the member is fully urging the tube bracket against the roller assembly. This provides an indication to the user that the peristaltic pump may be turned on/the system is ready to be used. For example the crest shape of the part may be in a vertical orientation, and the step of applying a force may include rotating the crest of the handle from a horizontal to a vertical orientation, such that the crest of the handle aligns with the crest of the part.

An eighth embodiment comprises a method for distributing liquid to a user, the method comprising: fitting a tube bracket onto a roller assembly of a peristaltic pump, wherein the tube bracket comprises a housing which defines a tube path, the tube path holding a tube for liquid communication between a liquid container and a medical applicator device; applying a force to a portion of the tube bracket to bias a portion of the tube against the roller assembly; and running the peristaltic pump to effect a flow of liquid from the liquid container to the medical applicator device.

The method of the eighth embodiment may comprise any or all of the optional features of the method of the seventh embodiment, or indeed any of the features of any of the other embodiments or optional features thereof.

A ninth embodiment comprises a method of replacing a tube in a liquid distribution system, the method comprising: removing a tube bracket fitted on a roller assembly of a peristaltic pump, the tube bracket having a tube received in a tube path thereof and the tube being removed therewith; fitting a new tube bracket onto the roller assembly wherein the new tube bracket comprises a new tube received in a tube path of the new tube bracket; and applying a force to a part of the new tube bracket configured to receive a force so as to bias a portion of the new tube against the roller assembly.

Fitting the new tube bracket may comprise simply placing the tube bracket over the roller assembly of the pump. Applying the force to a part of the new tube bracket configured to receive a force may comprise simply turning a handle to engage a member with a part of the tube bracket configured to receive a force (as discussed previously). Therefore the replacement of the tube bracket is a simple, easy task for the user.

Prior to removing the tube bracket the method may comprise removing a force being applied to a part of a tube bracket configured to receive a force so as to stop biasing a portion of the tube against the roller assembly of the peristaltic pump.

A tenth embodiment comprises a method of replacing a tube assembly according to the second embodiment or third embodiment in a liquid distribution system according to the fourth, fifth or sixth embodiments.

It will be well appreciated that the numerous features described with reference to the tube bracket, tube assembly or system are equally applicable to the methods described herein. Therefore, the methods may include using or operating any of the described features.

### BRIEF DESCRIPTION OF DRAWINGS

Certain embodiments of the technology will now be described, by way of example only, with reference to the accompanying drawings, in which:

FIG. 1 shows a tube assembly for a liquid distribution system;

FIG. 2 shows a liquid distribution system comprising the tube assembly of FIG. 1;

FIG. 3 shows a front and back perspective view of the tube bracket of the tube assembly of FIG. 1;

FIG. 4 shows a back view of the tube bracket;

FIG. 5 shows a perspective view of the tube bracket being fitted onto a roller assembly of a peristaltic pump to assemble the system of FIG. 2;

FIGS. 6a-6c show the liquid distribution system with a handle in different positions;

FIGS. 7a-7c are detailed views of the parts of FIGS. 6a-6c denoted by circles X, Y and Z respectively and show a mechanism of the peristaltic pump assembly which can be operated to engage the tube bracket with the roller assembly;

FIG. 8 is a detailed view of the part of FIG. 6c indicated by circle W and shows a view of the top part of the tube bracket in the system of FIG. 6c;

FIG. 9a shows the liquid distribution system indicating section line S-S; and

FIG. 9b shows a cross-section through the knob taken along section line S-S shown in FIG. 9a.

### DETAILED DESCRIPTION

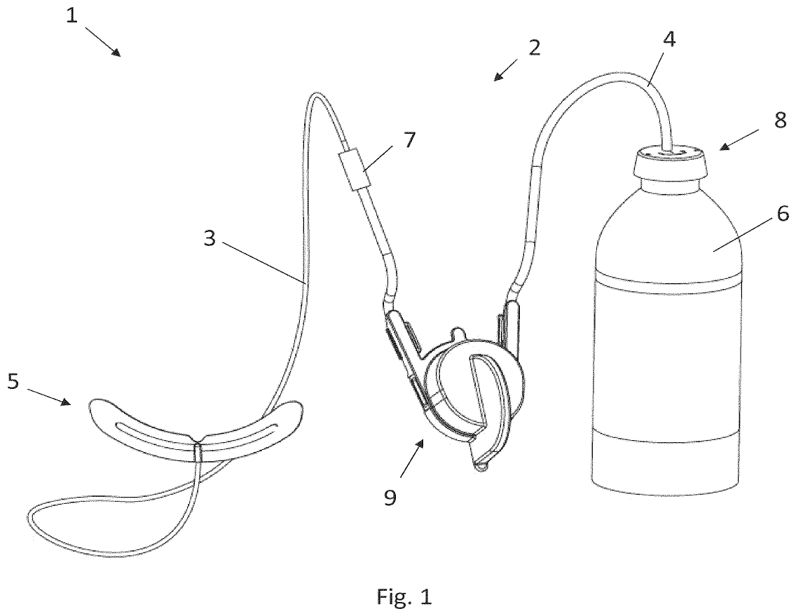

FIG. 1 shows a tube assembly 1 for a liquid distribution system. The tube assembly 1 comprises a tube 2 comprising a first tube section 3 and a second tube section 4.

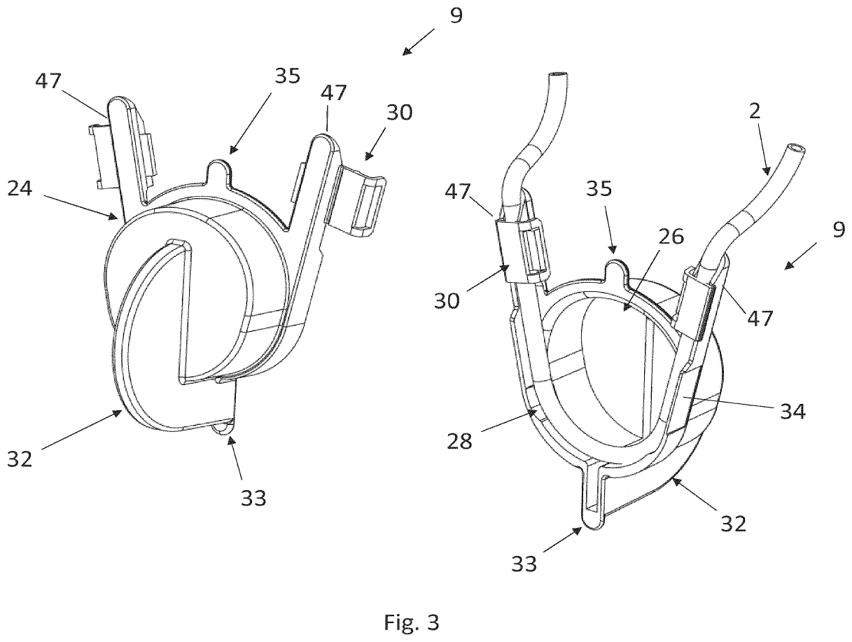

A portion of the second tube section 4 is secured in a tube bracket 9. Details of the tube bracket 9 are discussed later with reference to FIGS. 3 and 4.

In the described example, the first tube section 3 is connected to a mouthpiece 5 for a user's mouth. However, the first tube section 3 may be connected to any (medical) device suitable for administering liquid to a user. The second tube section 4 is connected to a container 6 containing liquid to be distributed to the mouthpiece 5, the tube 2 providing a means of liquid communication from the container 6 to the mouthpiece 5.

The first tube section 3 is joined to the second tube section 4 by a connector 7. The connector 7 may be a simple connector, or may be a check valve configured to inhibit liquid from the mouthpiece 5 flowing back into the second tube section 4 and/or the container 6. The second tube section 4 comprises tubing that is thicker than the tubing of the first tube section 3.

The container end of the tube 2 (e.g. the end of the second tube section 4 connected to the container 6) is secured in a seal in a screw top 8 for the container 6. Thus the tube 2 is configured to be easily attachable to and detachable from the container 6.

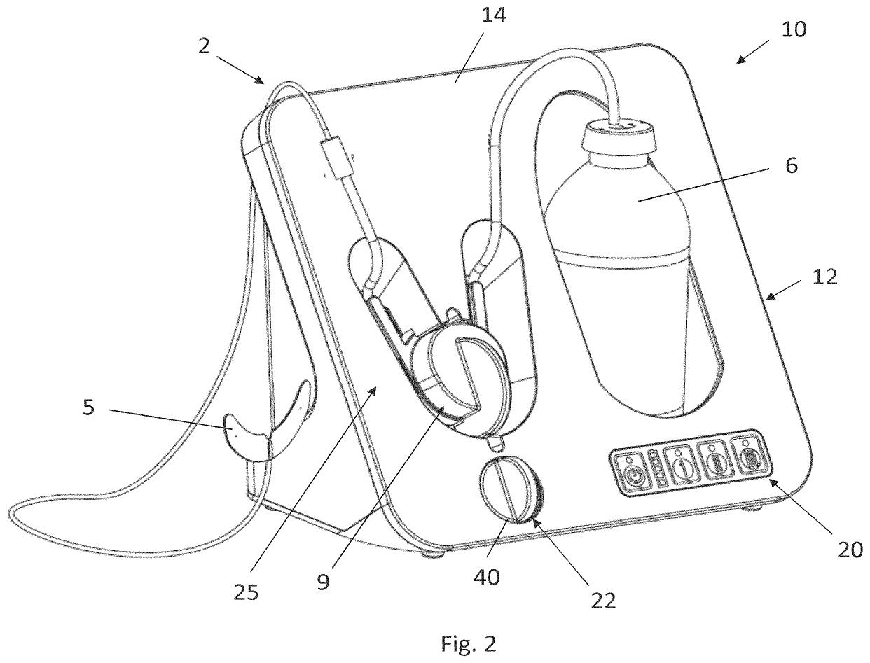

FIG. 2 shows a liquid distribution system 10. The system 10 comprises the tube assembly 1 and a peristaltic pump assembly 12 having a peristaltic pump 25. The peristaltic pump assembly 12 is configured for conducting the flow of liquid through the tube assembly 1. In other words, the peristaltic pump assembly 12 is configured to draw liquid from the container 6, through the tube 2, and to the mouthpiece 5.

The pump assembly 12 comprises a housing 14 configured to hold the inner workings of the pump assembly 12 such as a motor, control unit and other electronic circuitry (not shown). The housing 14 also comprises portions configured to support/hold features of the tube assembly 1, such as a receptacle configured to receive the container 6.

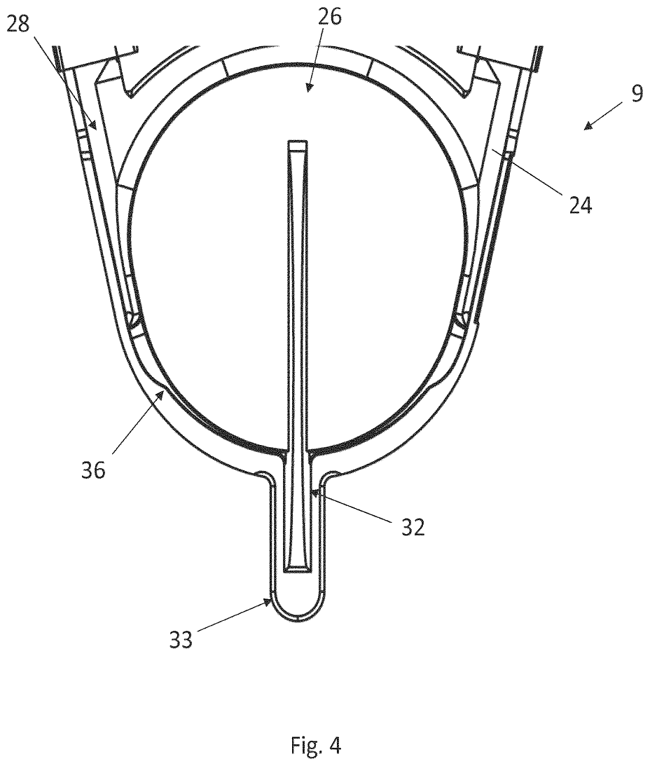

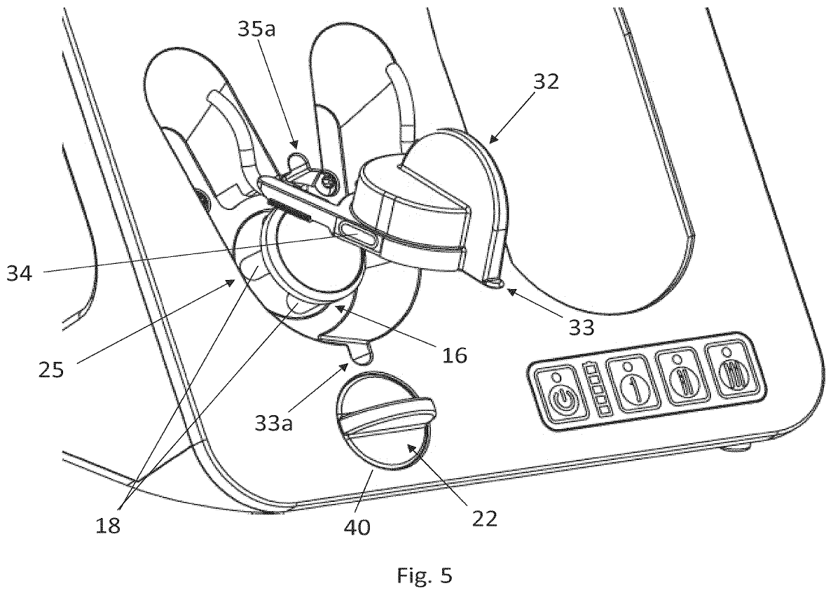

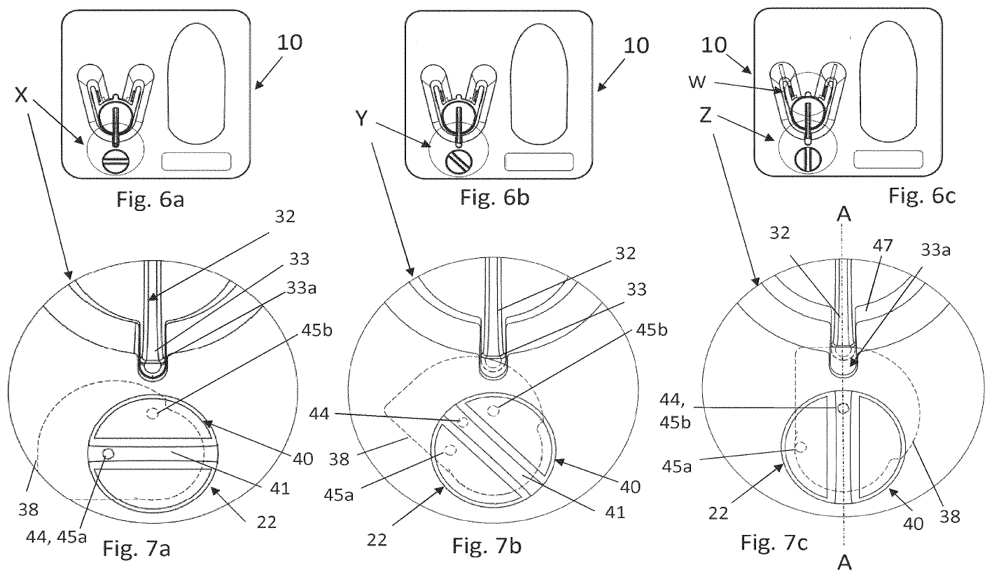

The pump 25 of the pump assembly 12 comprises a roller assembly 16 (see FIG. 5) operatively connected to the motor. With reference to FIGS. 5 and 6, the roller assembly 16 comprises four rollers 18 attached around its external circumference (typically the roller assembly comprises two or more rollers). The rollers 18 are configured to compress the tube 2 of the tube assembly 1 as they are brought into contact with the tube 2 by the rotation of the motor.

The pump assembly 12 comprises a control panel 20 by which the user may control the operation of the pump assembly 12. As shown in FIG. 2, the control panel 20 comprises a number of buttons, one or more buttons corresponding to a speed of the motor (e.g. a rotation speed of the roller assembly 16). The control panel 20 may, alternatively or in addition, comprise a dial or a touch screen for control of the pump assembly 12. The control panel 20 may be in communication with, or be part of, a control unit (not shown).

The pump assembly 12 comprises a force application mechanism 22 for applying a force to the tube bracket 9 and thereby biasing a portion of the tube 2 against the roller assembly 16. Specifically, the mechanism 22 is operable by a user to apply a force to the tube bracket 9 once the tube bracket 9 has been fitted into the pump assembly 12 (that is, when the tube bracket 9 is placed onto the roller assembly 16). The mechanism 22 urges the tube bracket 9 towards the roller assembly 16 and compresses a portion of the tube 2 against the rollers 18 of the roller assembly 16. In the example shown in FIG. 2, the mechanism 22 comprises a handle, in this case a knob 40 (in the form of a rotatable dial) having a grip 41 and a flange member 38 configured to engage the protrusion 32.

The mechanism 22 is shown in more detail in FIGS. 6a-6b, 7a-7b, 9a and 9b. FIGS. 6a-6c show the liquid distribution system 10 with the knob 40 in different positions. The buttons of the control panel 20 are omitted for simplicity. FIGS. 6a-6b show the knob 40 in horizontal, intermediate and vertical positions respectively. The horizontal position corresponds to a non-operational or disengaged state of the mechanism 22, the intermediate position corresponds to a partially engaged state of the mechanism 22, and the vertical position corresponds to an operational or fully engaged state of the mechanism 22. FIGS. 7a, 7b and 7c show a detailed view of the parts X, Y and Z of FIGS. 6a, 6b and 6c respectively.

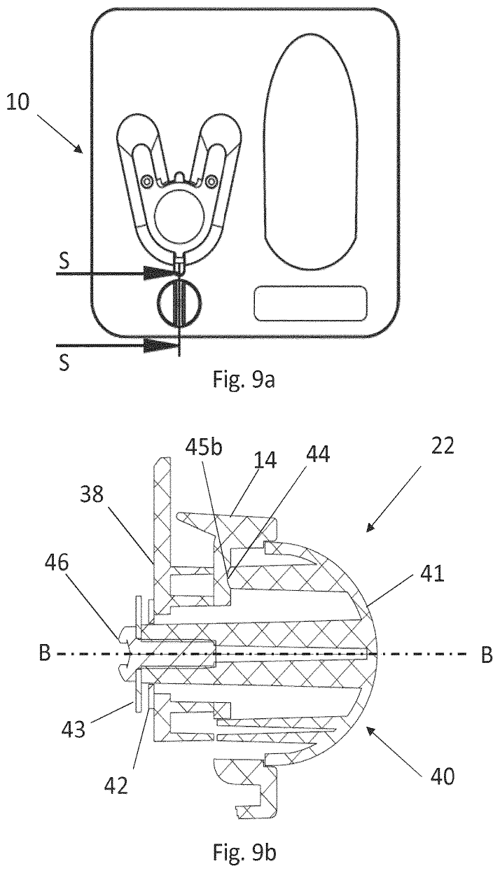

FIG. 9a shows the liquid distribution system 10 with a section line S-S marked thereon. FIG. 9b is the cross-section through the knot taken along section line S-S marked in FIG. 9a. As can be seen in FIG. 9b, the knob 40 is rotatably mounted on housing 45 by means of fastener (screw) 46. Flange 38 is coupled to the knob 40 and thus is rotatable therewith around axis B-B. A spring washer 42 is arranged around the fastener 46 and presses against the flange 38, which presses against the housing 14 and holds the knob 40 in place. However, it also allows some movement of the knob along axis B-B. A plain washer 43 is also arranged around the fastener 46 proximate the spring washer 42. If the knob 40 is pulled away from the housing 14 by the user, the plain washer will abut the spring washer 42 and transmit the force from the spring washer 42 to the fastener 46 and the knob 40, thus acting to move the knob 40 back along axis B-B to be seated against the housing.

The knob 40 also comprises a bulb-shaped member 44 with a complementary shape to two pockets 45a, 45b in the housing such that the bulb 44 can be seated therein. One of the pockets 45b can be seen in the cross-section of FIG. 9b, with bulb 44 seated therein. Both pockets 45a, 45b can be seen in FIGS. 7a-7c. Bulb 44 is arranged towards the edge of the knob 40 and aligned with the grip 41. It is located such that when the knob 40 is in the non-operational position illustrated in FIGS. 6a and 7a, the bulb 44 is located laterally horizontally to the left of the axis B-B; and when the knob 40 is in the operational position illustrated in FIGS. 6c and 7c, the bulb is located vertically above the axis B-B. Pocket 45a is arranged in the housing 14 so as to align with the bulb 44 when the knob is in the non-operational position such that the bulb 44 is seated in the pocket 45a; and pocket 45b is arranged in the housing 14 so as to align with the bulb 44 when the knob is in the operational position such that the bulb 44 is seated in the pocket 45b. The operation of the mechanism 22 will be described later.

Turning to FIGS. 3 and 4, the tube bracket 9 is shown in further detail. The tube bracket 9 comprises a housing 24 defining a pump cavity 26 and a tube path 28. The pump cavity 26 is configured for receiving the roller assembly 16. The tube path 28 is configured for receiving the tube 2 (particularly, the second tube section 4). The housing 24 has a pair of arms 47 The tube 2 is secured in the tube path 28 by a pair of clips 30.

The tube path 28 has portion which is generally arcuate and extends at least partially circumferentially around the pump cavity 26. In other words, the pump cavity 26 is generally circular in shape, and the tube path 28 extends around at least part (for example, between 25% and 50%) of the circumference of the pump cavity 26. The housing 24 has a pair of straight arms 47 extending away from the pump cavity 26, the tube path 28 extending along these arms. The clips 30 are located on these arms 47. The tube path 28 of one of the arms 47 guides the tube 2 towards the pump cavity 26 from the container 6 and the tube path 28 of one of the other arms 47 guides the tube 2 away from the pump cavity 26 to the mouthpiece 5. The tube bracket 9 comprises a part configured to receive a force, in this case a protrusion 32. The protrusion 32 extends outwardly from the housing 24 and is configured to receive a force applied to the tube bracket 9 such that, in use, a portion of the tube 2 held in the tube path 28 is biased against the roller assembly 16 positioned in the pump cavity 26. In other words, a force can be applied to the protrusion 32 in order to engage the tube 2 with the roller assembly 16 for effective operation of the peristaltic pump. As can be seen in the Figures, in this example the protrusion 32 comprises a crest shape. The protrusion 32, in particular the crest shape, also provides a convenient means for a user to grip the tube bracket 9 during installation.

The protrusion 32 of the tube bracket 9 comprises a tab 33. The protrusion tab (bracket tab) 33 is configured to fit into a corresponding slot 33a in the pump assembly 12 when the tube assembly 1 is installed.

Similarly, the housing 24 of the tube bracket 9 comprises a tab 35. The housing tab 35 is configured to fit into a corresponding slot 35a in the pump assembly 12 when the tube assembly 1 is installed. The slot 35a comprises a peripheral recess 35b extending into the housing 14 of the pump assembly into which the tab 35 can move when a force is applied to the tube bracket 9.

The tube bracket 9 includes a RFID tag 34 for identification of the tube bracket 9. The electronics of the pump assembly 12 comprises an RFID reader (not shown) positioned to read the RFID tag 34 of a tube bracket 9 fitted into the pump assembly 12. The RFID reader is configured to receive data from the RFID tag of the tube bracket 9, and identify if the tube bracket 9 (and its associated tube assembly 1) can be used (e.g. whether it is safe to use) with the pump assembly 12.

Although not shown in the example in the Figures, the container 6 may include an RFID tag for identification of the container 6. Accordingly, the electronics of the pump assembly 12 may comprise an RFID reader positioned to read the RFID tag 34 of the tube bracket 9 and/or an RFID reader positioned to read the RFID tag of the container 6.

As shown in FIG. 4, the tube path 28 comprises a shoulder portion 36. The shoulder portion 36 extends slightly into the tube path 28 and is configured to, in use, position the portion of the tube 2 to be biased closer to the roller assembly 16 than the rest of the tube 2 in the tube path 28.

Advantageously, the tube assembly 1 is a single-use, i.e. disposable, item that is easily replaceable in the system 10. Typically, the tube assembly 1 is used for up to 24 hours. At the end of that time, it would generally be disposed of and replaced with a new tube assembly 1. This provides the benefit that it is not necessary to clean the mouthpiece 5 and/or assembly 1 to maintain good hygiene. The simple design of the mouthpiece 5 and the tube assembly 1 makes it cost-effective to manufacture, whereby single use is feasible for the user and/or manufacturer.

A method of distributing liquid to a user, comprising assembling a system 10 from the tube assembly 1 and the pump assembly 12, will now be described, with reference to FIGS. 5, 6a-6c, 7a-7c, 8, 9a and 9b.

A user is provided with a replaceable tube assembly 1 as shown in FIG. 1. That is, the user is provided with the mouthpiece 5, container 6, and tube 2, wherein the tube 2 has been preassembled into the tube bracket 9 (and is secured by the clips 30). The container 6 is placed in the receptacle in the housing 14 of the pump assembly 12 and the tube bracket 9 is fitted into the pump assembly 12. That is, as shown in FIG. 5, the pump cavity 26 of the tube bracket 9 is placed over the roller assembly 16, and the protrusion tab 33 and housing tab 35 are inserted into respective slots 33a, 35a in the housing 14 of the pump assembly 12. The user can easily grip the tube bracket 9 by the protrusion 32 when installing it in the pump assembly 12. Once the tube bracket 9 has been installed in the pump assembly 12, the grip 41 of the knob 40 is operated by a user (i.e. turned by a user) to engage an arcuate portion of the member 38 with the protrusion 32 and to start urging the tube bracket 9 toward the roller assembly 16, in order to move the tube bracket 9 to an operational position. The edge of the arcuate portion presses against the protrusion 32 as it rotates and gradually pushes the tube bracket 9 upwards. FIGS. 6a-6c and corresponding FIGS. 7a-7c show the knob 40 being turned from a non-operational position to an operational position. As can be seen in FIG. 7a, when the knob is turned fully horizontally in the non-operational position, the flange 38 is completely disengaged from the protrusion 32. Here, the bulb 44 is seated in pocket 45a in the housing and this engagement maintains the knob 40 in that position. When a user applies a force to turn the knob clockwise, the force applied by the user forces the bulb 44 out of pocket 45a (which is possible because the knob 40 can move outwards along axis B-B) and thus allows the knob 40 to turn. As the knob 40 is turned clockwise, the flange 38 abuts the protrusion 32 (FIG. 7b) and gradually pushes the tube bracket 9 upwards. Once the knob 40 reaches the vertical operational position the flange 38 has pushed the tube bracket 9 a maximal amount so that the tube bracket 9 is in a fully operational configuration. A gap 47 between the housing 14 and the tube bracket 9 is now at its largest. In this position the bulb 44 is aligned with pocket 45b, and the force exerted by spring washer 42 against plain washer 43 acts to move the knob 40 back towards the housing such that bulb 44 is seated in pocket 45b. This engagement maintains the knob 40 in this operational position. The user can feel the bulb 44 disengaging/engaging with the pockets 45a, 45b (with a “snap”) and thus can feel when the knob 40 is in a non-operational/operational position. The flange 38, bulb 44 and pockets 45a, 45b are shown in dashed lines to indicate they are hidden in use.

As can be seen in FIGS. 7b and 7c, when the member 38 is in contact with the protrusion 32, the member 38 covers the protrusion tab 33 in the slot 33a. This restricts the movement of the tab 33 in the slot 33a and helps prevent the tube bracket 9 from being removed from the pump assembly 12 (e.g. accidentally by the user).

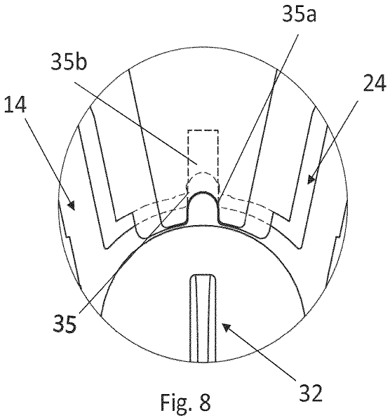

Similarly, as shown in FIG. 8, as the tube 2 in the tube bracket 9 is urged toward the roller assembly 16, the housing tab 35 is gradually inserted into the recess 35b which is formed in slot 35a. The housing tab 35 is thus locked into the recess 35b, which helps prevent the tube bracket 9 from being removed from the pump assembly 12 (e.g. accidentally by the user). FIG. 8 shows tab 35 located in the recess 35; the tab 35 and recess 35b are in dashed lines to indicate they are hidden behind the front of the housing 14.

With reference to the example shown in FIG. 7c, the grip 41 comprises a crest shape configured to be aligned with the crest shape of the protrusion 32 when the member 38 is in a maximal position. In other words, when the member 38 is fully urging the tube bracket 9 against the roller assembly 16, the crest shape of the grip 41 is aligned with the crest of the protrusion 32 (aligned along axis A-A in FIG. 7c). This, in addition to the feeling provided by the engagement/disengagement of the bulb 44 with the pockets 45a, 45b, provides an indication to the user that the peristaltic pump 12 may be turned on/the system 10 is ready to be used. Furthermore, in this position a flat portion of the edge of the member 38 is engaged with the protrusion 32 which is a stable configuration that assists in maintaining the mechanism 22 in an operational position even after the user releases the grip 41. Therefore, the member 38 maintains a force on the protrusion 32 by virtue of both the flat portion of the flange 38 and the bulb 44/pockets 45a, 45b. Whilst both these means are used to maintain the force on the protrusion 32 in the illustrated example, in other examples only one of these means may be used to maintain the force after the user has released the grip 41. Accordingly, the tube bracket 9 can be installed in the peristaltic pump assembly 12, and the liquid distribution system 10 set up, by a series of straightforward, easy steps even for those who do not possess good fine motor skills.

Once it has been assembled, the liquid distribution system 10 is operated using the control panel 20 to control the speed of the pump (i.e. the rotation speed of the roller assembly 16) and thus control the volume flow rate of liquid to the mouthpiece 5.

After a predetermined period of use, the control unit of the liquid distribution system 10 may determine that part or all of the tube assembly 1 (particularly, for example, the tube bracket 9 and/or the container 6) needs to be replaced. For instance, the control unit may receive information from the RFID reader of the pump assembly 12 positioned to read the RFID tag 34 of the tube bracket 9, and may identify that a usage lifetime of the tube bracket 9 (e.g. 24 hours) has elapsed. Alternatively, the control unit may determine that the volume of liquid in the container is low, and should therefore be replaced, by using the RFID reader of the pump assembly 12 positioned to read the RFID tag of the container 6 to determine the volume of liquid initially in the container 6, and calculating the volume of liquid that has been delivered to the mouthpiece 5 by the pump.

If part or all of the tube assembly 1 needs to be replaced, the liquid distribution system 10, by means of the control unit, will alert the user. For example, the liquid distribution system 10 may comprise an alarm or an LED (not shown) in order to alert the user when the replaceable tube assembly 1 should be replaced with another.

1. A tube bracket for operatively engaging a tube with a peristaltic pump, the tube bracket comprising:
a housing defining:
a pump cavity for receiving a roller assembly of the peristaltic pump; and
a tube path for receiving the tube, the tube path extending at least partially circumferentially around the pump cavity; and
a part configured to receive a force such that, in use, a portion of the tube received in the tube path is biased against the roller assembly of the peristaltic pump in the pump cavity.

2. A tube bracket as claimed in claim 1, wherein the tube path comprises a shoulder portion configured to, in use, position the portion of the tube to be biased closer to the roller assembly than the rest of the tube.

3. (canceled)

4. A tube bracket as claimed in claim 1, wherein the part configured to receive a force is integrally formed with or attached to the housing such that the part configured to receive a force cannot move relative to the housing.

5. A tube bracket as claimed in claim 1, wherein the part configured to receive a force comprises a protrusion extending outwardly from the housing.

6. A tube bracket as claimed in claim 1, comprising a bracket tab extending outwardly in a radial direction of the pump cavity, wherein the bracket tab is configured to fit into a slot of a housing of the peristaltic pump when the tube bracket is fitted onto the roller assembly of the peristaltic pump.

7. A tube bracket as claimed in claim 1, wherein the tube bracket housing comprises a housing tab extending outwardly in a radial direction of the pump cavity, wherein the housing tab is configured to fit into a slot of a housing of the peristaltic pump when the tube bracket is fitted onto the roller assembly of the peristaltic pump.

8. A tube assembly for a liquid distribution system, the tube assembly comprising:
a tube for liquid communication; and
a tube bracket as claimed in claim 1.

9. A tube assembly as claimed in claim 8, wherein the tube is preassembled in the tube bracket.

10.-11. (canceled)

12. A liquid distribution system comprising:
a medical applicator device for administering liquid to a user;
a container for containing the liquid to be distributed to the medical applicator device;
a tube assembly as claimed in claim 8, wherein the tube is fluidly connected to the container and the medical applicator device; and
a peristaltic pump assembly for effecting a flow of the liquid from the container to the medical applicator device;
wherein the tube bracket is fitted onto a roller assembly of the peristaltic pump assembly.

13. A system as claimed in claim 12, wherein the peristaltic pump assembly comprises a mechanism for applying a force to the part of the tube bracket configured to receive a force so as to bias a portion of the tube against the roller assembly of the peristaltic pump assembly.

14. A system as claimed in claim 13, wherein the mechanism comprises a member configured to engage the part of the tube bracket configured to receive a force and to apply a force thereto.

15. A system as claimed in claim 14 wherein the member is configured to be locked in a position of engagement with the part of the tube bracket configured to receive a force so as to maintain the force applied thereto.

16. A system as claimed in claim 14, wherein the mechanism comprises a handle coupled to the member, and wherein the handle can be operated to adjust the force applied to the part,
wherein the handle is rotatably mounted to a housing of the pump assembly;
wherein the member is rotatably mounted to a housing of the pump assembly such that the member is rotatable between a position in which the member does not apply a force to the part of the tube bracket configured to a receive a force and a position in which the member applies a force to the part of the tube bracket configured to receive a force; and
wherein the member comprises an arcuate portion configured to adjust the force applied to the part upon rotation of the handle.

17.-19. (canceled)

20. A system as claimed in claim 14, wherein the tube bracket comprises a bracket tab extending outwardly in a radial direction of the pump cavity and wherein the bracket tab is fitted into a bracket tab slot of the housing of the peristaltic pump assembly; and
wherein, the member is mounted to the pump assembly such that, when the member is engaged with the part configured to receive a force, the member restricts movement of the bracket tab in the bracket tab slot.

21. A system as claimed in claim 14, wherein the tube bracket housing comprises a housing tab extending outwardly in a radial direction of the pump cavity and wherein the housing tab is fitted into a housing tab slot of the housing of the peristaltic pump assembly; and
wherein the housing tab slot comprises a peripheral recess and the system is configured such that, as the force applied to the part configured to receive a force is increased, the bracket tab moves into the recess.

22. A method of distributing liquid to a user, the method comprising:
providing a medical applicator device for administering liquid to a user, a container for containing the liquid to be distributed to the medical applicator device, and a tube assembly as claimed in claim 8, wherein the tube is fluidly connected to the container and the medical applicator device;
providing a peristaltic pump assembly for effecting a flow of the liquid from the container to the medical applicator device;
fitting the tube bracket of the tube assembly onto a roller assembly of the peristaltic pump assembly;
applying a force to the part configured to receive a force to bias a portion of the tube of the tube assembly against the roller assembly; and
running the peristaltic pump assembly to effect the flow of liquid from the container to the medical applicator device.

23. A method as claimed in claim 22, wherein applying a force to the part configured to receive a force so as to bias a portion of the tube against the roller assembly comprises operating a mechanism of the peristaltic pump assembly to urge the part configured to receive a force toward the roller assembly.

24. A method as claimed in claim 23, wherein the mechanism comprises a handle coupled to a member configured to engage the part configured to receive a force and to apply a force thereto, and operating the mechanism comprises operating the handle to adjust the force applied to the part configured to receive a force by the member.

25. A method as claimed in claim 24, wherein the member comprises an arcuate portion configured to, in response to rotation of the handle, engage the part configured to receive a force and adjust the force applied to the part configured to receive a force.

26. A method of replacing a tube in a liquid distribution system, the method comprising:
removing a tube bracket fitted on a roller assembly of a peristaltic pump, the tube bracket having a tube received in a tube path thereof and the tube being removed therewith;
fitting a new tube bracket onto the roller assembly wherein the new tube bracket comprises a new tube received in a tube path of the new tube bracket; and
applying a force to a part of the new tube bracket configured to receive a force so as to bias a portion of the new tube against the roller assembly.
